(19) United States
(12) Patent Application Publication
(10) Pub. No.: US 2023/0183246 A1
(43) Pub. Date: Jun. 15, 2023

(54) CONTINUOUS FLOW SONOGASHIRA COUPLING SYNTHESIS METHOD

(71) Applicant: Purdue Research Foundation, West Lafayette, IN (US)

(72) Inventors: David Harley Thompson, West Lafayette, IN (US); Herman O. Sintim, Westfield, IN (US); Qingqing Qi, West Lafayette, IN (US); Shruti Biyani, West Lafayette, IN (US)

(73) Assignee: Purdue Research Foundation, West Lafayette, IN (US)

(21) Appl. No.: 17/926,379

(22) PCT Filed: May 13, 2021
(86) PCT No.: PCT/US21/32143

§ 371 Date: Nov. 18, 2022

Related U.S. Application Data
(60) Provisional application No. 63/026,771, filed on May 19, 2020.

(51) Int. Cl. C07D 487/04; C07D 401/06; C07D 471/04; B01J 31/24

(52) U.S. Cl. CPC C07D 487/04; C07D 401/06; C07D 471/04; B01J 31/2404; B01J 2531/824; B01J 2231/32

(57) ABSTRACT

The present disclosure relates to a telescoped continuous flow Sonogashira coupling synthesis for some lead compounds to support in vivo studies and pre-clinical evaluation. The application of high throughput tools combined with the telescoped continuous synthesis method can enable an efficient and safe synthesis of compounds of interest involving hazardous coupling reagents such as HATU, while minimizing by-product formation.

### GOVERNMENT RIGHTS

This invention was made with government support under Award No. W911NF-16-2-0020 awarded by the Army Research Lab and Award No. CA023168 awarded by the National Institutes of Health. The government has certain rights in the invention.

### TECHNICAL FIELD

The present disclosure relates to a telescoped continuous flow Sonogashira coupling synthesis method for some lead compounds to support in vivo studies and pre-clinical evaluation.

### BACKGROUND

This section introduces aspects that may help facilitate a better understanding of the disclosure. Accordingly, these statements are to be read in this light and are not to be understood as admissions about what is or is not prior art.

In spite of the growing interest in continuous synthesis of new chemical entities by the pharmaceutical industry, the application of design of experiments (DoE) strategies with guidance from high throughput experiments (HTE) for reaction optimization remains an underdeveloped area. Rapid discovery of the most favorable and benign conditions for integration into continuous synthesis schemes can also enable the development of efficient and robust synthetic routes, potentially reducing the time required for translating a medicinal chemistry lead through clinical trials. One such medicinal chemistry challenge is acute myeloid leukemia (AML), an aggressive form of blood cancer that remains difficult to treat despite tremendous efforts by academics and pharmaceutical companies to find a durable cure. The 5-year survival rate for AML is approximately 30% and drops below 10% in patients over 65 years, highlighting the dire need for a better treatment. Fms-like tyrosine kinase 3 (FLT-3) is a class of receptor tyrosine kinase that is overexpressed in AML patients. Nearly one third of initially responding patients have shown relapse over time by developing secondary mutations during treatment. Sintim and coworkers discovered HSN608 (Scheme 1, compound 5) as a lead compound with high potency toward inhibition of all FLT-3 forms. This lead also displays inhibition activity of RET and its mutant forms better than the reported RET inhibitors. In order to support the drug development effort to enable in vivo experiments and toxicology studies for this lead compound, identification of a synthetic route that can be rapidly upscaled is essential.

The batch synthesis reported for HSN-608 had several drawbacks, including high catalyst and co-catalyst loadings of 10 mol % Pd and 5 mol % Cu, respectively, low yield, use of an explosive reagent, HATU, for amidation that would be unsafe for large scale batch synthesis, and a long reaction time of ˜24 hour for the two-steps (FIG. 1). We sought to improve the safety, efficiency, and heat & mass transfer profile of this synthesis by executing it in flow. Furthermore, continuous flow methods also provide a rapid reaction optimization platform to enable reductions in catalyst loadings that otherwise contribute to high production costs and Pd-associated toxicity in active pharmaceutical ingredients. Reduced batch variability, smaller reactor footprint, and minimized solvent waste will also render the overall process greener. The aim of this study is to identify the optimal conditions for synthesis of HSN-608 utilizing microfluidics with the assistance of high throughput experimentation tools. Once the optimized synthesis is identified, it can be further upscaled to support preclinical studies of this lead agent.

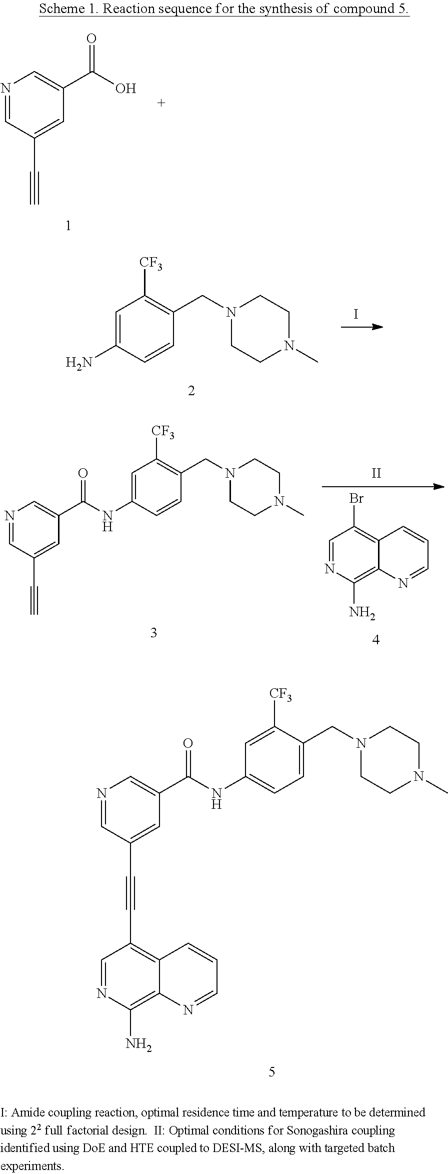

The one variable at a time (OVAT) approach only provides local knowledge of reactivity patterns and are, therefore, blind to the interaction between variables. The DoE approach is not subject to this limitation, making it ideal for studying interrelated variables. HTE utilizing a robotic liquid handling system provides a platform to integrate automation with synthesis that facilitates interrogation of a wide array of reaction condition variables simultaneously. While conventional LC-MS takes up to 33 hours for analysis of 384 reactions, desorption electrospray ionization mass spectrometry (DESI-MS) is capable of analyzing up to 6144 reactions on a single plate array in less than 2.5 h without any requirement for work-up. The large data set of full mass spectra at each point in the array that is generated by this method enables rapid searches for the m/z of interest and follow up MS/MS experiments using automated Chemical Reaction Integrated Screening software (CHRIS) for target and/or byproduct structure confirmation. Streamlining of our workflow in this manner allowed us to quickly identify crucial reaction parameters that informed our understanding of the reaction mechanism in the key C—C bond forming step. Using these powerful new reaction discovery tools (FIG. 1), we report a strategy for expediting the optimization of transformations involved in the two-step synthesis of a target molecule in flow using DoE, HTE, and DESI-MS for identification of a continuous flow synthetic route.

Given the importance of cross-coupling reactions in medicinal chemistry, Sonogashira transformations in flow have been reported, however, the heteroatom density of our target molecule compared to other reported target molecules makes the transition metal catalyzed flow synthesis of 5 challenging.

### SUMMARY

The present disclosure relates to a telescoped continuous flow Sonogashira coupling synthesis for some lead compounds to support in vivo studies and pre-clinical evaluation.

In one embodiment, the present disclosure provides a continuous flow Sonogashira coupling synthesis method to prepare a compound of Formula I, wherein the method comprises:

providing a first flow comprising an aryl or a heteroaryl halide compound of Formula II, a base, and an optional Cu(I) halide;

providing a second flow comprising a Pd(II)-based catalyst;

providing a third flow comprising an aryl or heteroaryl alkyne of Formula III;

wherein the first flow, second flow and the third flow are mixed and fed into a flow reactor to carry out a Sonogashira coupling reaction to provide the compound of Formula I,

wherein the Formula I is

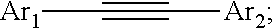

wherein the Formula II is Ar₁—X, where X is Cl, Br, or I;

wherein the Formula III is

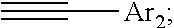

wherein Ar₁ and Ar₂ are each independently an optionally substituted monocyclic or bicyclic aryl or heteroaryl.

In one embodiment the present disclosure provides a continuous flow Sonogashira coupling synthesis method to prepare a compound of Formula A, wherein the method comprises:

providing a first flow comprising an aryl or a heteroaryl halide compound of Formula B, a base, and an optional Cu(I) halide;

providing a second flow comprising a Pd(II)-based catalyst;

providing a third flow comprising an aryl or heteroaryl carboxylic acid of Formula C, an aryl or heteroaryl amine of Formula D; and

providing a fourth flow comprising an amide coupling reagent,

wherein said third flow and fourth flow are mixed and fed into a first reactor to carry out an amide coupling reaction to provide an amide compound of Formula E, and the formed amide compound of Formula E is released from the first reactor to form a fifth flow,

wherein the first flow, second flow and the fifth flow are mixed and fed into a second reactor to carry out a Sonogashira coupling reaction to provide the compound of Formula A,

wherein the Formula A is:

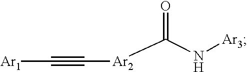

wherein the Formula B is Ar₁—X, X is Cl, Br, or I;

wherein the Formula C is:

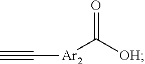

wherein the Formula D is Ar₃—NH₂,

wherein the Formula E is:

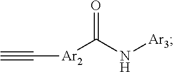

wherein Ar₁, Ar₂, Ar₃ are each independently an optionally substituted monocyclic or bicyclic aryl or heteroaryl.

### BRIEF DESCRIPTION OF THE DRAWINGS

FIG. 1 shows conventional batch reaction method (A) vs. an approach utilizing DoE/HTE/DESI-MS→flow strategy for rapid discovery of favorable conditions for flow synthesis of compound 5 (B). HTE using DESI-MS readout to guide DoE in batch and flow can also provide mechanistic information. Taken together, these data enable the rapid development of efficient synthesis conditions.

FIG. 2 shows the microfluidic synthesis of 3 via HATU-mediated amide coupling in a glass reactor chip (staggered oriented ridge i.e. SOR mixer chip 3227 by Chemtrix) where A: 0.277 M 1 & 2 (1 equiv.) and DIPEA (3 equiv.) in DMF and B: HATU in DMF (1.1 equiv.), Pressure: Ambient pressure, Reactor volume: 19.5 μl.

FIG. 3 shows contour plot of residence time, temperature and isolated yield from the HATU-mediated amide coupling using a 2² full factorial DoE evaluation. Yield for the reaction is color-coded where dark red indicates highest yield while dark blue indicates the lowest yield regime.

FIG. 4 shows HTE heat map for Sonogashira coupling indicating product ion counts in each cell (mean of four reaction replicates where the number corresponds to MS ion count from DESI-MS). Time: 90 minutes, 240 minutes (indicated in light pink), Temperature 100° C., 150° C. (dark pink), five different bases at 1.5 equiv. (aqua green) and 3 equiv. with TEA (aqua green), 6 solvents, and two stoichiometries (1:1 and 1:2.5 (blue)). The scale based on the product ion count is shown using the color in the arrow where dark red represents highest product ion count (more efficient or successful reaction), while white represents lower product ion count (unsuccessful reaction). An ion count of 100 is considered as the threshold and cells with >100 are considered as successful reactions.

FIG. 5 shows interaction plots between Time (90 minutes, 240 minutes), Temperature (100° C., 150° C.), and Stoichiometry (1:1, 1:2.5), where the response is indicated on the y-axis as the DESI-MS product ion counts obtained from the 2³ full factorial design experiment. The legend corresponding to the specific conditions are depicted below the individual graphs. A. The blue line represents 90 minutes and orange line represents 240 minutes. B & C: The blue line represents stoichiometry 1:1 and orange line represent stoichiometry 1:2.5. In case of higher stoichiometry, alkyne 8 was used in excess.

FIG. 6 shows a contour plot of time and temperature vs. product ion counts to identify the optimal reaction time and temperature. The mean product ion count values of four replicates for the case of EtOH as solvent and TEA as base was used. The color-coded scale is used for the Product Ion Count as the response from DESI-MS. Dark red represents highest ion count (most optimal reaction condition) with blue representing the lowest ion counts (least favorable reaction condition). The plot was generated using the statistical software, Minitab where X=Temperature, Y=Time and Response=Product Ion Count. For 20° C., the reaction mixture was pinned directly at 90 minutes and 240 minutes without any heating.

FIG. 7 shows global analysis and trends for 288 unique Sonogashira reactions at 55° C. from HTE using 3 and 4 as substrates at m/z=546.2. Xphos Pd G3 was used as the catalyst, with average product ion counts calculated from ion counts measured for reactions that were pinned onto the DESI plate in duplicate. The three grids refer to the three solvents DMAC, DMF, NMP with fill colors representing the ligand condition (Pink=Brett Phos, Blue=Ru Phos, Green=No additional ligand). Each column point represents one of four different time points (0 h is when the solution from the 384 well was pinned immediately onto the PTFE plate while the other time points are 1.5 h, 6.75 h, 12 h at 55° C.). Each row of conditions corresponds to the additives: 7 bases and one ligand (in case of [P(t-Bu)₃H]BF₄. The size of the bubble corresponds to the average product ion count from DESI-MS where larger diameter spots represent higher ion counts, thus representing more efficient reaction conditions.

FIG. 8 shows a DESI-MS HTE plot of Sonogashira coupling for the synthesis of HSN-608. The reaction conditions were Xphos Pd G3, DMF, 1.5 h, 55° C. Structures for the 7 bases and 1 ligand are shown. Additional ligand conditions are color coded for each type (Pink=Brett phos, Green=No ligand, Purple=Ru phos). The red dashed line represents a signal-to-noise ratio of 3 as the cutoff. Noise signals were measured by taking the average at a region on the PTFE plate where no reaction mixture was pinned. Product ion counts above this cut-off are considered as a successful reaction at m/z=546.2 [5+H] with higher ion counts corresponding to a more efficient reaction condition.

FIG. 9 shows Telescoped continuous flow synthesis of HSN608. f₁, f₂, f₃, f₄ are the flow rates. Residence time for amide coupling: 20 minutes, 50° C. and T_{R2}: Residence time for Sonogashira coupling 2 h, 4 h, 6 h at 95° C.

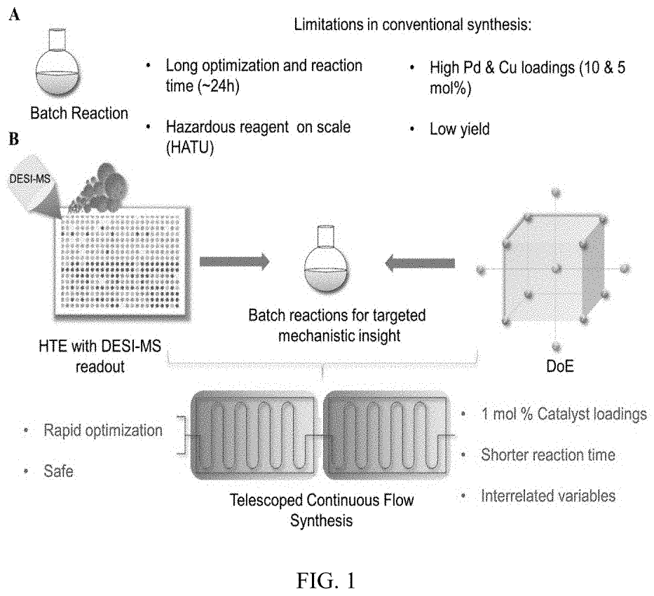

### DETAILED DESCRIPTION

For the purposes of promoting an understanding of the principles of the present disclosure, reference will now be made to the embodiments. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

Non-limiting examples of substituents, that can be bonded to a substituted carbon (or other such as nitrogen) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen or a carbon-based moiety, and wherein the carbon-based moiety can itself be further substituted; for example, wherein R can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R can be independently mono- or multi-substituted; or wherein two R groups bonded to a nitrogen atom or to adjacent nitrogen atoms can together with the nitrogen atom or atoms form a heterocyclyl, which can be mono- or independently multi-substituted.

The term “alkyl”, alone or in combination, signifies a straight-chain or branched-chain alkyl group with 1 to 8 carbon atoms, preferably a straight or branched-chain alkyl group with 1 to 6 carbon atoms and particularly preferred a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, preferably methyl and ethyl and most preferred methyl.

The term “cycloalkyl”, alone or in combination, signifies a cycloalkyl ring with 3 to 8 carbon atoms and preferably a cycloalkyl ring with 3 to 6 carbon atoms. Examples of C₃-C₈ cycloalkyl are cyclopropyl, methyl-cyclopropyl, dimethyl-cyclopropyl, cyclobutyl, methyl-cyclobutyl, cyclopentyl, methyl-cyclopentyl, cyclohexyl, methyl-cyclohexyl, dimethyl-cyclohexyl, cycloheptyl and cyclooctyl, preferably cyclopropyl and particularly cyclopentyl.

The term “heteroaryl” represents aromatic ring comprising at least one hetero atom such as N, S, O, or Se. Hetero aryl in the present disclosure may be any hetero aryl. Hetero aryl in the present disclosure may be but is not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, benzimidazolinyl groups, naphthyridine (such as 1, 7 naphthyridine), or any combination thereof.

In one embodiment, the present disclosure provides a continuous flow Sonogashira coupling synthesis method to prepare a compound of Formula I, wherein the method comprises:

providing a first flow comprising an aryl or a heteroaryl halide compound of Formula II, a base, and an optional Cu(I) halide;

providing a second flow comprising a Pd(II)-based catalyst;

providing a third flow comprising an aryl or heteroaryl alkyne of Formula III;

wherein the first flow, second flow and the third flow are mixed and fed into a flow reactor to carry out a Sonogashira coupling reaction to provide the compound of Formula I,

wherein the Formula I is

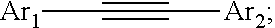

wherein the Formula II is Ar₁—X, where X is Cl, Br, or I;

wherein the Formula III is

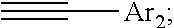

wherein Ar₁ and Ar₂ are each independently an optionally substituted monocyclic or bicyclic aryl or heteroaryl.

In one embodiment regarding to the continuous flow Sonogashira coupling synthesis method to prepare a compound of Formula I, wherein the Pd(II)-based catalyst comprises PdCl₂(PPh₃)₂, PdCl₂(MeCN)₂, XPhos Pd G3, PdCl₂, allyl palladium(II) chloride dimer, Pd(amphos)Cl₂, or Na₂PdCl₄.

In one embodiment regarding to the continuous flow Sonogashira coupling synthesis method to prepare a compound of Formula I, wherein the base comprises pyrrolidine, K₂CO₃, Cs₂CO₃, 2,2,6,6 tetramethyl piperidine, tetrabutylammonium hexafluorophosphate, 1,4-diazabicyclo[2.2.2]octane (DABCO), quinuclidine, triethyl amine (TEA), N,N-diisopropylethylamine (DIPEA), tetrabutylammonium acetate, or piperidine.

In one embodiment regarding to the continuous flow Sonogashira coupling synthesis method to prepare a compound of Formula I, wherein the optional Cu(I) halide is CuI.

In one embodiment regarding to the continuous flow Sonogashira coupling synthesis method to prepare a compound of Formula I, wherein each flow comprises a solvent, wherein the solvent comprises N-methyl-2-pyrrolidone (NMP), dimethylformamide (DMF), DMF/H₂O, ethanol, dimethylacetamide (DMAC), N, N′-dimethylpropyleneurea (DMPU), 1,4-dioxane, tetrahydrofuran (THF), or 2-methyl tetrahydrofuran.

In one embodiment regarding to the continuous flow Sonogashira coupling synthesis method to prepare a compound of Formula I, wherein each flow comprises an optional ligand, wherein the ligand comprises [(t-Bu)₃PH]BF₄, t-Bu₃P, RuPhos, Brett Phos, or XPhos.

In one embodiment regarding to the continuous flow Sonogashira coupling synthesis method to prepare a compound of Formula I, wherein one or more hydrogens of Ar₁ and/or Ar₂ can be independently substituted by —NH₂, —CN, —CF₃, —F, Cl, —Br, —I, —OH, or an optionally substituted amide, sulfonamide, or urea.

In one embodiment regarding to the continuous flow Sonogashira coupling synthesis method to prepare a compound of Formula I, wherein the flow reactor is a plug flow reactor, segmented flow reactor, a microreactor, coiled tubing reactor, coiled flow inverter (CFI) reactor, or a continuous stirred tank reactor (CSTR) in a flow configuration.

In one embodiment regarding to the continuous flow Sonogashira coupling synthesis method to prepare a compound of Formula I, wherein the flows are mixed using T-mixers, Y-mixers, static-mixers, ultrasonic mixers, staggered oriented ridge mixers, zig-zag mixers, packed bed mixers, CFI mixers, or paddle mixers in a CSTR configured for flow synthesis.

In one embodiment the present disclosure provides a continuous flow Sonogashira coupling synthesis method to prepare a compound of Formula A, wherein the method comprises:

providing a first flow comprising an aryl or a heteroaryl halide compound of Formula B, a base, and an optional Cu(I) halide;

providing a second flow comprising a Pd(II)-based catalyst;

providing a third flow comprising an aryl or heteroaryl carboxylic acid of Formula C, an aryl or heteroaryl amine of Formula D; and

providing a fourth flow comprising an amide coupling reagent,

wherein said third flow and fourth flow are mixed and fed into a first reactor to carry out an amide coupling reaction to provide an amide compound of Formula E, and the formed amide compound of Formula E is released from the first reactor to form a fifth flow,

wherein the first flow, second flow and the fifth flow are mixed and fed into a second reactor to carry out a Sonogashira coupling reaction to provide the compound of Formula A,

wherein the Formula A is:

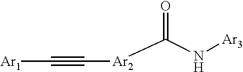

wherein the Formula B is Ar₁—X, X is Cl, Br, or I;

wherein the Formula C is:

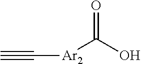

wherein the Formula D is Ar₃—NH₂,

wherein the Formula E is:

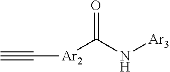

wherein Ar₁, Ar₂, Ar₃ are each independently an optionally substituted monocyclic or bicyclic aryl or heteroaryl.

In one embodiment regarding to the continuous flow Sonogashira coupling synthesis method to prepare a compound of Formula A, wherein the Pd(II)-based catalyst comprises PdCl₂(PPh₃)₂, PdCl₂(MeCN)₂, XPhos Pd G3, PdCl₂, allyl palladium(II) chloride dimer, Pd(amphos)Cl₂, or Na₂PdCl₄.

In one embodiment regarding to the continuous flow Sonogashira coupling synthesis method to prepare a compound of Formula A, wherein the base comprises pyrrolidine, K₂CO₃, Cs₂CO₃, 2,2,6,6 tetramethyl piperidine, tetrabutylammonium hexafluorophosphate, 1,4-diazabicyclo[2.2.2]octane (DABCO), quinuclidine, triethyl amine (TEA), N,N-diisopropylethylamine (DIPEA), tetrabutylammonium acetate, or piperidine.

In one embodiment regarding to the continuous flow Sonogashira coupling synthesis method to prepare a compound of Formula A, wherein the optional Cu(I) halide is CuI.

In one embodiment regarding to the continuous flow Sonogashira coupling synthesis method to prepare a compound of Formula A, wherein each flow comprises a solvent, wherein the solvent comprises N-methyl-2-pyrrolidone (NMP), dimethylformamide (DMF), DMF/H₂O, ethanol, dimethylacetamide (DMAC), N, N′-dimethylpropyleneurea (DMPU), 1,4-dioxane, tetrahydrofuran (THF), or 2-methyl tetrahydrofuran.

In one embodiment regarding to the continuous flow Sonogashira coupling synthesis method to prepare a compound of Formula A, wherein each flow comprises an optional ligand, wherein the ligand comprises [(t-Bu)₃PH]BF₄, t-Bu₃P, RuPhos, Brett Phos, or XPhos.

In one embodiment regarding to the continuous flow Sonogashira coupling synthesis method to prepare a compound of Formula A, wherein one or more hydrogen of Ar₁, Ar₂ and/or Ar₃ can be independently substituted by —NH₂, —CN, —CF₃, —F, Cl, —Br, —I, —OH, or an optionally substituted amide, sulfonamide, or urea.

In one embodiment regarding to the continuous flow Sonogashira coupling synthesis method to prepare a compound of Formula A, wherein the flow reactor is a plug flow reactor, segmented flow reactor, microreactor, coiled tubing reactor, coiled flow inverter (CFI) reactor, or continuous stirred tank reactor (CSTR) in a flow configuration.

In one embodiment regarding to the continuous flow Sonogashira coupling synthesis method to prepare a compound of Formula A, wherein the flows are mixed using T-mixers, Y-mixers, static-mixers, ultrasonic mixers, staggered oriented ridge mixers, zig-zag mixers, packed bed mixers, CFI mixers, or paddle mixers in a CSTR configured for flow.

In one embodiment regarding to the continuous flow Sonogashira coupling synthesis method to prepare a compound of Formula A, wherein Ar₁ is

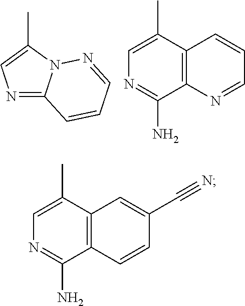

Ar₂ is a pyridinyl ring, and

### Ar₃ is

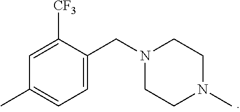

In one embodiment regarding to the continuous flow Sonogashira coupling synthesis method to prepare a compound of Formula A, wherein the compound of Formula A is selected from:

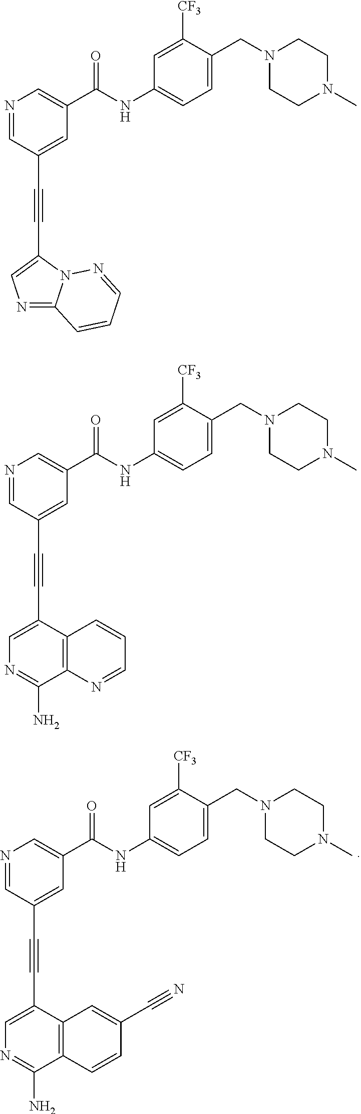 [SMILES: CN1CCN(Cc2ccc(NC(=O)c3cncc(C#Cc4cnc5cccnn45)c3)cc2C(F)(F)F)CC1 ; CN1CCN(Cc2ccc(NC(=O)c3cncc(C#Cc4cnc(N)c5ncccc45)c3)cc2C(F)(F)F)CC1 ; CN1CCN(Cc2ccc(NC(=O)c3cncc(C#Cc4cnc(N)c5ccc(C#N)cc45)c3)cc2C(F)(F)F)CC1]

In one embodiment regarding to the continuous flow Sonogashira coupling synthesis method to prepare a compound of Formula A, wherein the compound of Formula A is:

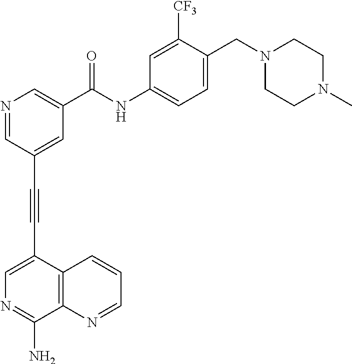 [SMILES: CN1CCN(Cc2ccc(NC(=O)c3cncc(C#Cc4cnc(N)c5ncccc45)c3)cc2C(F)(F)F)CC1]

Results and Discussion

DoE for Amide Coupling in Flow. Our initial batch experiments employed a number of common amide coupling reagents, however, these typically resulted in poor yields (<40%). The highest isolated yields of ˜55-60% were observed in small-scale batch reactions with HATU. Since reaction scale up with this explosive reagent would be a safety concern, we pursued a safer alternative by developing a HATU-mediated amide coupling process in flow. Although the use of HATU in flow reactions has been reported for peptide synthesis, we sought to quickly identify optimal conditions for its use in the preparation of intermediate 3.

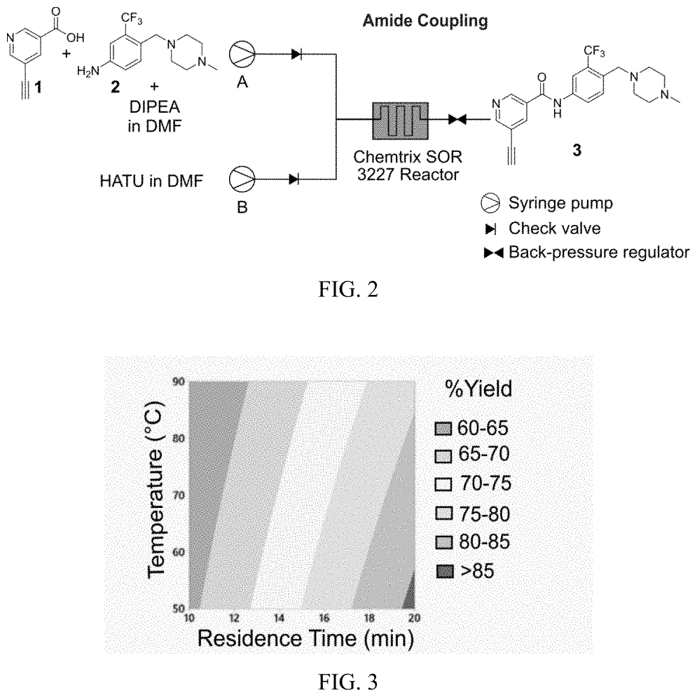

We chose DMF as the solvent since it is a low volatility, high boiling polar aprotic solvent with high dielectric constant although on scale it does have reproductive toxicity as an exposure concern. Our initial flow experiments did not use an in-line mixer (without T-mixer in FIG. 2). HATU in DMF was mounted on one syringe port and a mixture of 1+2+DIPEA in DMF solutions were injected directly at 50° C. inside a glass reactor chip via the second port. Very low product formation was observed due to formation of guanidino derivatives as observed by (+) ESI-MS. We then changed the set-up and employed a micro-static mixer prior to the chip that we hypothesized would allow for the formation of activated ester at 20° C. using 0.378 M solutions of the reagents. However, clogging was observed due to the relatively low solubility of HATU (1.1 equiv.) in DMF. Next, we incorporated a T-mixer (FIG. 2) at 20° C. to allow for the formation of activated ester before all the reactants entered the glass reactor chip held at 50° C. using 0.277 M reagent concentrations. After confirming intermediate 3 formation by (+) ESI-MS and observing the product profile in the crude mixture by LC-MS, we decided to quickly identify the most favorable conditions using a 2² full factorial design. We hypothesized that this approach would allow us to identify high yielding conditions in a minimum number of experiments. The reaction times chosen were 10 and 20 minutes and temperatures of 50° C. and 90° C. FIG. 3 summarizes the yield contours for 3 obtained from the 2² full-factorial experiment.

A mid-point experiment at 75° C. for 15 minutes was also investigated; a 65% conversion was observed in this case. Since amide coupled product isolation by column chromatography was tedious, we developed a precipitation method for reaction work-up wherein water addition at the end of the reaction produces a solid form of 3 that can be easily recovered by filtration in ˜86% yield (50° C., R_{T}=20 min). A further upscaling of this method to a simple coiled tube reactor in a 50° C. oil-bath enabled the rapid synthesis of intermediate 3 (2.260 g total) at a rate of 96 mg/h.

Discovering Conditions for Sonogashira Coupling Using Automation (DoE, HTE, and DESI-MS Experiments). Based on reports of other Pd-catalyzed reactions, we expected that the order of reagent addition could be an important parameter for efficient execution of the Sonogashira transformation, particularly when using a robotic open air liquid handling system. Our first set of experiments focused on identifying the best order of addition and an initial screen of catalysts. We performed this experiment on model substrates 6 and 8 (Scheme 2) for four different orders of addition and two different catalysts, Xphos Pd G3 and Pd(OAc)₂, at 70° C. in ACN. Analysis of 96 unique reactions with 4 replicates was performed via HTE and analyzed using (+) ESI-MS, running a script in Perl software to generate Excel data files as previously reported. We found that Xphos Pd G3 gave the greatest number of hits. These data also revealed that extended contact of the base and catalyst at room temperature during liquid transfer without the reactants present did not yield the desired product, likely due to catalyst degradation since Pd(0) has lower stability in the presence of air—the conditions existing while the reaction mixtures were being built by the liquid handling robot.

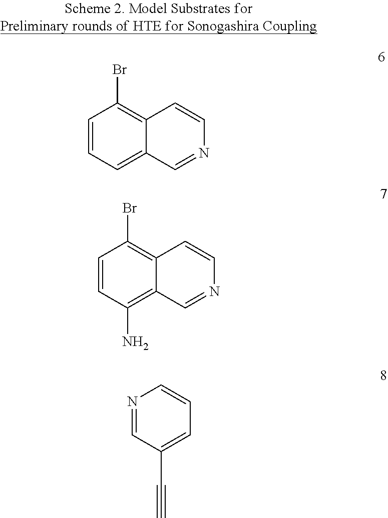

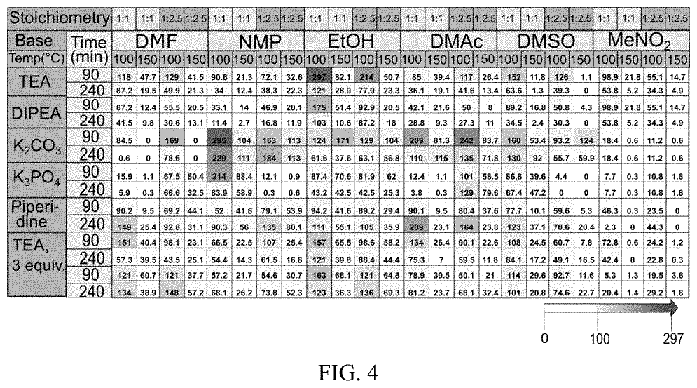

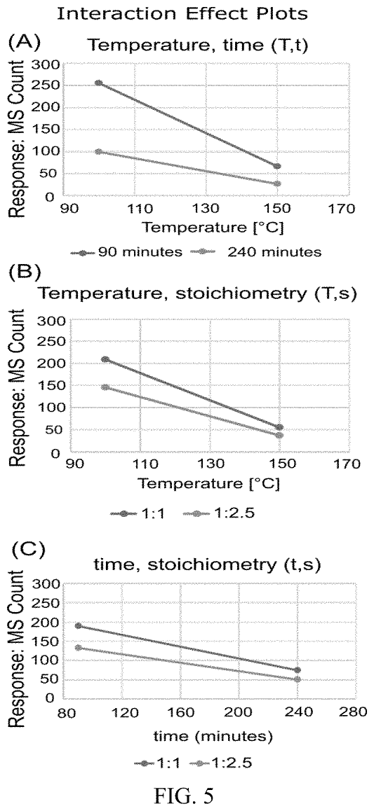

In the second HTE campaign, our goal was to identify the most crucial continuous variables and to understand the interaction between the variables. We evaluated 5-bromo-8-aminoisoquinoline 7 and 3-ethynylpyridine 8 as model reactants (Scheme 2), given their structural similarity to 4 and 3, respectively. We chose a 2³ full factorial DoE approach coupled with a DESI-MS readout, where time, temperature, and stoichiometry were chosen as the main HTE factors. The low and high levels were: time=90 and 240 min, temperature=100 and 150° C., and stoichiometry=1:1 and 1:2.5 (where 8 was in excess). This DoE was performed using six different solvents (DMF, NMP, DMSO, nitromethane, DMAc, and EtOH) and five different bases (K₂CO₃, K₃PO₄, piperidine, TEA, and DIPEA) to produce 336 unique reactions on a single DESI-MS plate (FIG. 4). We had also transferred 30 μL of the reaction mixture at 20° C. after mixing all the reagents in a 96 well block before transfer in quadruplicate to a 384 well-plate for pinning onto a PTFE DESI plate at 90 minutes and 240 minutes. The product ion counts from the DESI-MS analysis were taken as the output to make the interaction plots (FIG. 5), identify the most crucial variable, perform analysis of variance (ANOVA) and develop Pareto plots.

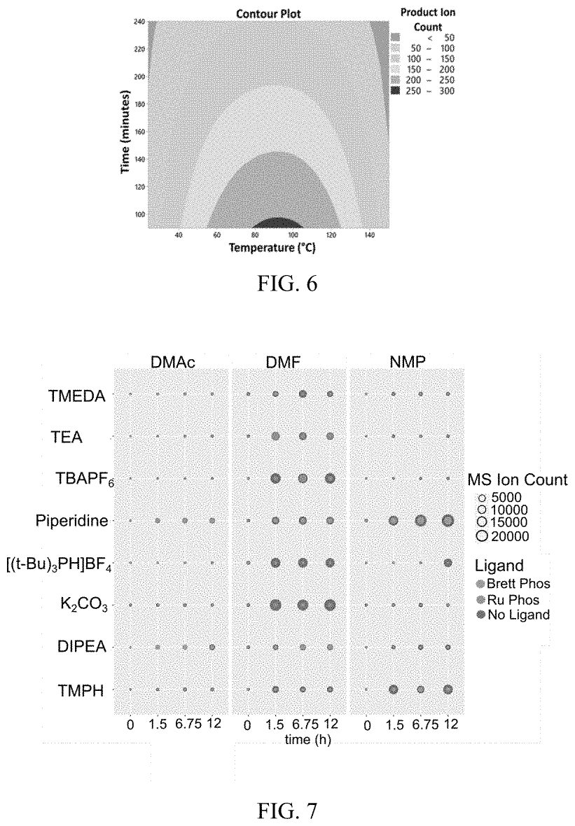

We observed significant hits with solvent/base combinations as indicated in the heat map in FIG. 4, including EtOH/TEA, NMP/K₂CO₃, NMP/K₃PO₄, and DMAc/piperidine. All the reaction mixtures were pinned onto the same PTFE plate for DESI-MS analysis. We observed that temperature had the greatest impact on reaction efficiency (FIG. 5) with lower temperature (100° C.) particularly favoring higher product ion intensities. Higher reaction temperatures (150° C.) likely results in catalyst deactivation since palladium catalyst oxidation has been reported in the literature at elevated temperature. The second most important parameter revealed is the interaction between time and temperature, while stoichiometry had little to no impact on reaction efficiency. We concluded from FIG. 5 and the Pareto plot that the temperature, time and their interaction were the three most crucial variables. Thus, we then generated a contour plot (FIG. 6) that considered only these three variables and along with the ion counts we obtained at 20° C. so that we could identify the most favorable temperature for Sonogashira coupling. Using the contour plot based on the DESI-MS product ion counts, we found the optimal temperature range to be 78°-103° C., with reaction initiation occurring at ≥55° C. (FIG. 6).

After performing these high throughput experiments with the model substrates, we decided to move towards the continuous flow synthesis of the model substrates followed by HSN-608 synthesis directly in flow. We performed the continuous flow synthesis with model substrates using a glass microchip flow reactor mounted on a Labtrix S1 system (Chemtrix, Ltd., Netherlands) and monitored product formation by (+) ESI-MS. Having observed the Sonogashira coupling product for the model substrates at the respective m/z values from flow synthesis, we planned to directly translate these findings to HSN-608 and perform the optimization in flow. We investigated the flow synthesis of 5 using 3, 4, and K₂CO₃ in NMP as these gave us hits in the HTE (FIG. 4) and had also worked for the model substrate. However, we did not see product 5 by (+) ESI-MS even though the coupling product was observed for the model compounds in flow. The poor solubility of 4 in EtOH also limited our options in microfluidics. Therefore, we pursued another round of HTE to identify the discrete variable most favorable for the synthesis of 5.

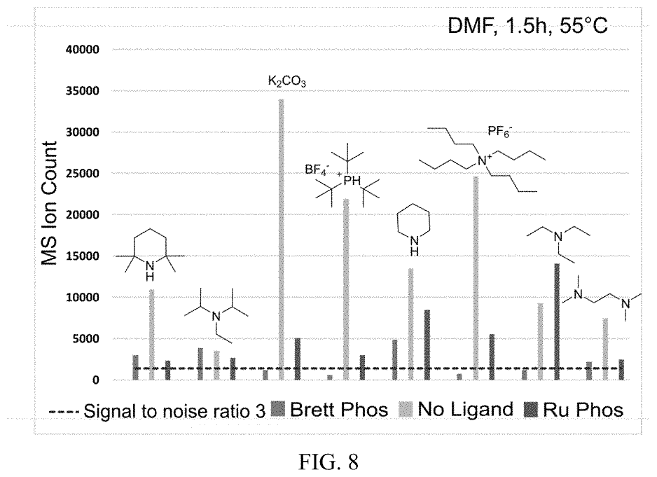

Based on the findings from the previous DoE showing that the reaction becomes kinetically favorable at ≥55° C. (FIG. 6), we performed the next HTE at 55° C. The aim of this round was to identify the most favorable solvent, base, and ligand. We chose seven different bases/salts with pKₐ values ranging between 10.25 to 11.40: TMEDA, TEA, TBAPF₆, piperidine, DIPEA, K₂CO₃, 2,2,6,6-tetramethylpiperidine (TMPH), and a ligand [(t-Bu)₃PH]BF₄. We limited our experiment to three solvents (DMF, NMP, and DMAc) due to poor solubility of 4 in other solvents. We also added RuPhos, and BrettPhos to the HTE since these ligands reportedly enhance oxidative addition and reductive elimination. The overall results are shown in FIG. 7. Reactions in DMF were found to provide the widest choice of base over NMP and DMAc. Interestingly, reactions with no additional ligand showed higher reaction efficiency than those loaded with RuPhos or BrettPhos (bigger circle size for green as indicated in FIG. 7). More favorable bases are the ones with higher pKₐ values like piperidine, TMPH, and salts like K₂CO₃, TBAPF₆, and the ligand [(t-Bu)₃PH]BF₄ (FIG. 7 and FIG. 8). Since base is crucial for deprotonation to form the Cu-acetylide intermediate (Scheme 3), we inferred from these findings (FIG. 8) that the formation of this intermediate and thus Cycle B is a key step in the reaction and that enhancement of the transmetalation step would likely lead to further increases in the reaction efficiency.

Translating HTE Findings to Better Understand the Coupling Mechanism of Compounds 3 & 4 in Batch and Flow. In order to test our conclusion that Cycle B is crucial as inferred from our HTE results, we performed two sets of experiments in batch mode (Table 1) where we monitored the completion of the reaction by TLC at different time points. Reaction completion was taken in this initial study as disappearance of 3 by TLC analysis. We used 5-bromo-8-amino-1,7-naphthyridine (4) and 5-iodo-8-amino-1,7-naphthyridine (9) in the Sonogashira coupling using different catalysts and bases. We found that the reaction was complete after 12 h with aryl bromide 4 but was not complete even after 15 h with aryl iodide 9. We interpret these findings to mean that the two cycles are not well synchronized with aryl iodide 9 such that cycle A proceeds faster than cycle B, leading to an increase in the decomposition products from cycle A as evidenced by more spots on TLC and faster consumption of 9. In contrast, when the corresponding aryl bromide was used, the two cycles A & B were better synchronized, resulting in more of the desired product.

TABLE 1
Sonogashira time-to-completion experiments with
alkyne 3 in batch mode.

| | | | | | Time^{a} |
|---|---|---|---|---|---|
| Entry | Halide | Catalyst | CuI | Base | (h) |
| 1 | 4 | PdCl₂(PPh₃)₂/ | Yes | TMPH | 12^{b} |
| | | XPhos | | | |
| 2 | 9 | PdCh(PPh₃)₂/ | Yes | TMPH | >15^{b} |
| | | XPhos | | | |
| 3 | 4 | XPhos Pd G3 | Yes | Piperidine | 1 |
| 4 | 4 | [PdCl(allyl)]₂/ | No | Piperidine | >4 |
| | | [(t-Bu)₃PH]BF₄ | | | |
| 5 | 4 | Pd(amphos)Cl₂ | No | Cs₂CO₃ | >4 |

Reaction done at 80° C. unless otherwise mentioned
^{a}Time for complete consumption of 3 was monitored by TLC.
TMPH = 2,2,6,6 tetramethylpiperidine.
^{b}55° C.

Since oxidative addition rates are known to follow R-Br<R-I, we concluded that this step in the Sonogashira reaction is not the rate limiting step in the formation of 5. Further, we compared Cu-free reactions and reactions with 3% CuI and found that it takes >4 h to complete the reaction without Cu(I), but the reaction is completed in 1 h in the presence of Cu(I). We inferred from these studies, and in agreement with literature, that the transmetalation process was likely the slow step in the reaction and required further optimization (Scheme 3).

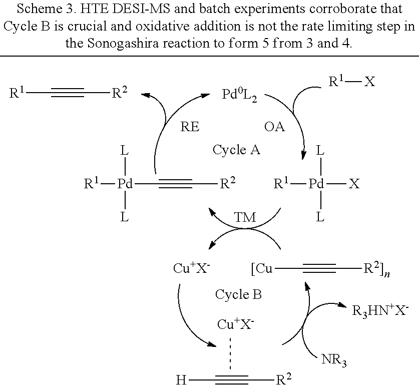

We then adapted these initial findings to flow where the first challenge was to address the relatively poor solubility of 4 (14.6 mg/ml, 0.065 M at 20° C. in DMF). Using a syringe heater in the reactor set-up solved the problem (solubility of 4: 38.1 mg/ml, 0.170 M at 55° C. in DMF), but as it would require an additional heating device for the tubing and syringe, we focused on 4 at 20° C. by recrystallizing it twice, first from EtOH and then from EtOAc. This treatment enabled a solubility increase to 0.0785 M (17.6 mg/ml in DMF). The reaction set-up used a T-mixer at 20° C. prior to injecting the solution of 4 into the heated glass reactor chip that was held at 80 or 95° C. Using TLC and LC-MS for reaction analysis, we found that the time required for both batch and flow transformations were the same (˜60 min) for complete conversion of 3. We did not attempt to accelerate the reaction rate by increasing the temperature above 100° C. since this would lead to catalyst degradation as suggested by FIG. 6, with subsequent reactor chip clogging. Based on these findings, we sought to modify our strategy for execution of this reaction in flow.

TABLE 2
Catalyst, ligand, & base selection for Sonogashira coupling
of 3 and 4 in flow using LC-MS to monitor product yield.
DMF as solvent, 3% Pd catalyst, 3% [(t-Bu)₃PH]BF₄ and 3%
CuI loadings (Rr = 30 min) were used unless otherwise noted.

| | | Temp | | % |
|---|---|---|---|---|
| Entry | Catalyst/Ligand | (° C.) | Base | Yield |
| 1 | PdCl₂/[(t-Bu)₃PH]BF₄ | 80 | Pyrrolidine | 10 |
| 2 | PdCl₂/[(t-Bu)₃PH]BF₄ | 95 | Pyrrolidine | 49 |
| 3 | PdCl₂(MeCN)₂/[(t-Bu)₃PH]BF₄ ^{a} | 80 | Cs₂CO₃ | 27 |
| 4 | PdCl₂(MeCN)₂/[(t-Bu)₃PH]BF₄^{b} | 80 | Pyrrolidine | 88 |
| 5 | PdCl₂(PPh₃)₂ | 80 | Pyrrolidine | 56 |
| 6 | PdCl₂(PPh₃)₂^{c} | 95 | Pyrrolidine | 85 (79) |
| 7 | PdCl₂(PPh₃)₂^{d} | 95 | Pyrrolidine | 73^{e} |

Isolated yield indicated in brackets.
^{a} DMF: H₂O.
^{b}6% catalyst, 4% CuI, 6% ligand.
^{c}1% catalyst, 1% CuI.
^{d}0.5% catalyst, 0.5% CuI.
^{e}Time for >90% conversion of 3 is ~80 min under batch conditions.

Synchronizing the Two Cycles to Enhance Reaction Efficiency. Given our findings that the transmetalation rate needed acceleration, we sought to increase synchronization of Cycles A and B in Scheme 2 by enhancing both the oxidative addition and Cu-acetylide formation steps simultaneously. To enhance the oxidative addition, we used the electron donating ligand [(t-Bu)₃PH]BF₄ and catalyst systems like PdCl₂(PPh₃)₂ and PdCl₂(MeCN)₂ as shown in Table 2. Furthermore, pyrrolidine was chosen as base due to its relatively high pKₐ11.27 to enhance Cu-acetylide formation as higher ion count was observed in HTE with base having higher pKₐ values (piperidine, TMPH). K₂CO₃ was not chosen due to its poor solubility in DMF. We observed an increase in yield with increasing temperature, consistent with our DoE findings (Entry 6). LC-MS analysis of the crude reaction mixture (Table 2) revealed lower amounts of by-product formation using PdCl₂(PPh₃)₂ as catalyst compared to PdCl₂/[(t-Bu)₃PH]BF₄, with >90% conversion of 3 seen in 30 min. Lowering the catalyst loadings to 0.5% increased the time required for the reaction more than 2-fold. Thus, we chose 1% PdCl₂(PPh₃)₂/pyrrolidine in DMF at 95° C. as our optimal condition for the telescoped flow synthesis of HSN608.

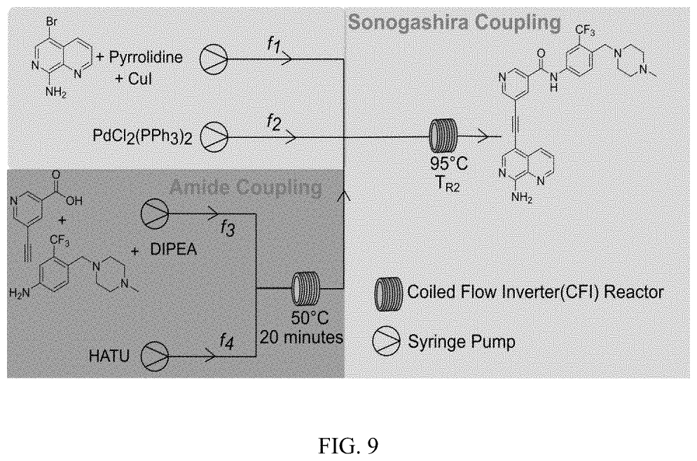

Telescoped Continuous Flow Synthesis of HSN608. We built a modular flow reactor comprised of a 4-way cross-assembly, two Harvard syringe pumps mounted with 25 mL syringes, and coiled flow inverter (CFI) reactors made from PFA tubing as shown in FIG. 9 for our telescoped flow synthesis efforts.

Reactions run at 0.075 M 4 caused deposition of solid in the syringe over time, stalling the pump within 8 h (Table 3, Entry 1). Lowering the concentration of 4 to 0.065 M overcame this problem. We then focused our effort on determining the optimal residence time for the second step in the telescoped reaction.

TABLE 3
Flow reactor conditions evaluated in the telescoped synthesis
of HSN608 using the reactor configuration shown in FIG. 6.

| Entry | f₁, f₂, f₃, f₄ (ul/min) | T_{R2} (h) | # CFI^{a} | % Yield^{b} |
|---|---|---|---|---|
| 1 | 14.26, 4.326, 4.45, 4.45^{c} | 2 | 1 | Pump stalled |
| 2 | 14.26, 4.326, 4.45, 4.45 | 2 | 1 | 15 |
| 3 | 14.26, 4.326, 4.45, 4.45 | 6 | 3 | 30 |
| 4 | 7.13, 2.16, 2.225, 2.225 | 4 | 2 | 54 |
| 5 | 7.13, 2.16, 2.225, 2.225^{d} | 4 | 2 | 32 |

[4] = 0.065M unless otherwise noted.
R_{T1} (amide coupling) = 20 min, with 1% PdCl₂(PPh₃)₂ and 1% CuI in the second step.
^{a}Number of CFI connected in series.
^{b}Isolated overall yield.
^{c}[0.075M].
^{d}0.5% PdCl₂(PPh₃)₂ and 0.5% CuI.
Reaction done at 95° C.

This optimization was achieved by connecting the CFI in series, producing an overall yield of 54% when lower flow rates and two CFI reactors in series were used (Entry 4). We learned for the telescoped flow reaction synthesis that lowering the catalyst loadings from 1 mol % to 0.5 mol % can reduce the yield from 54% to 32% (Entry 4 and 5). Furthermore, an optimal residence time, achieved by connecting appropriate number of the CFI in series, is crucial for an efficient telescoped flow synthesis. A plausible explanation for this is that the lower residence time resulting from fewer CFI (Entry 2) will not achieve a complete conversion of alkyne 3 and produce lower yields. Conversely, a larger number of CFI (Entry 3) can possibly introduce oxygen infusion from the surroundings between the connections, contributing to the formation of homocoupling by-product and reducing the yield of the desired product. By balancing these factors, we have identified a telescoped flow synthesis route for synthesis of 5 at 8 mg/h with continuous operation at >55 hours to enable the telescoped synthesis of HSN608. Overall, we have shown that the use of high throughput tools and a design of experiments approach, along with targeted bath mechanistic experiments, were able to rapidly guide the optimization and identification of a telescoped continuous flow synthesis of the potent FLT-3 inhibitor, HSN608. Scale-up of this route will be crucial to further evaluate the in vivo efficacy and toxicology of this lead compound. The capability of these tools can also be leveraged for developing efficient syntheses of other lead compounds in order to support drug discovery efforts. The flow methodology developed in this study can also be used for synthesis of other kinase inhibitors with similar structure motifs like ponatinib.

We have shown that a 2² full factorial design can be used to quickly optimize HATU-mediated amide coupling and enable safer handling of this explosive reagent on scale using a microfluidic flow reactor. A simple precipitation workup was also developed that circumvents the use of column chromatography for purification of 3. This work also demonstrates that a strategy employing a Design of Experiments/High Throughput Experimentation approach, coupled to desorption electrospray ionization-mass spectrometry, can rapidly identify the most favorable batch and flow conditions for Sonogashira coupling. These studies revealed the optimal choice of base, solvent and temperature conditions, as well as identifying that Cycle B is a key rate limiting step in the formation of 5. These findings focused our workflow towards synchronizing the two cycles through catalyst and base screening under flow conditions to enhance the reaction efficiency. We then applied these findings to identification of a continuous flow synthesis route for 5, a clinical candidate for the potential for treatment of acute myeloid leukemia. The catalyst and co-catalyst loadings were reduced by a 10-fold and 5-fold, respectively, along with improvements in the overall yield. These findings show that DoE coupled with HTE DESI-MS provides an opportunity for rapid optimization of synthetic transformations compared to conventional trial-and-error batch methods. Efforts are now underway to upscale the continuous synthesis of 5.

Experimental Section

Reagents. All reagents except 5-bromo-8-amino-1,7-naphthyridine (4) & 5-iodo-8-amino-1,7-naphthyridine (9) were purchased and used without further purification.

NMR Analysis. Samples for ¹H-NMR & ¹³C-NMR were analyzed by a Bruker AV-III-500-HD NMR spectrometer.

High Throughput Experiment. The HTE experiments were performed using a Biomek i7 liquid handling robot (Beckman Coulter Inc., Indianapolis). All the methods prepared utilized transfer of reagents dissolved in the desired solvent from chemically resistant reservoirs to the heating block. The span-8 pod in the robotic system, comprising of 8-channels to deliver the desired volumes from source to destination, was used. Pipetting techniques used in the robot for transfer of volatile solvents was modified to dispense accurate volumes. A multichannel pod on the robotic system comprised of 384-tip head was used to transfer from 96 to 384 well-plate and for mixing prior to heating the reactions. The heating blocks were brought to room temperature prior to pinning using the pin-tool onto the PTFE-coated DESI plate. The pinning method described previously for the transfer of 50 nL volumes using the pin-tool onto the porous PTFE surface was used.

Mass Spectrometry. (a) Electrospray Ionization-Mass Spectrometry (for identifying the order of addition in high throughput experiment and determination of the yield from continuous flow synthesis) was performed using a Thermo Fisher TSQ Quantum Access MAX mass spectrometer. This tool was connected to a Dionex Ultimate 3000 Series Pump and WPS-3000 Autosampler. An auto-sampler capable of handling 96 well plates was used for the analysis with a vapor-tight seal on the 96 well-plate. Data recording was achieved using parameters optimized for the ESI source, wherein the spray solvent was ACN with 0.1% formic acid or MeOH with 0.1% formic acid, spray voltage +5 kV (positive mode), capillary temperature 250° C., injection volume 1-5 μL. For the analysis and processing of data, Thermo Fisher Xcalibur software was utilized. (b) DESI-MS Analysis was performed using a previously published method¹⁷, except that samples at a density of 3072/plate and 1536/plate were used instead of the reported 6144/plate. In order to collect the DESI-MS data, we used a linear ion trap mass spectrometer (LTQ XL; Thermo Scientific) along with a DESI-imaging source (DESI 2D, Prosolia Inc.). Data acquisition was done using the software Xcalibur v3.0. The spray solvent used was MeOH or MeOH with 0.1% formic acid. The data generated is collected in the form of ‘yes/no’ output for each spot using an in-house built software to generate a heatmap and excel files. The average product ion counts from the Excel file generated for the Sonogashira coupling were used for the DoE Analysis in Minitab and for other analysis either using excel or R programming. Minitab is a user-friendly statistical software that helps to perform analysis and evaluation of statistics by providing the data as input. It also helps in the identification of trends and patterns to decipher and extrapolate solutions to a dataset containing problem. The response or Excel sheets can be directly used in the Minitab enabling the investigator to perform Analysis of Variance (ANOVA) Analysis, generate contour plots, and Pareto charts after choosing the input, output, and the response.

Microfluidic Synthesis. All the microfluidic experiments for screening of Sonogashira coupling and amide coupling reaction conditions were carried out using Chemtrix Labtrix S1 (Chemtrix, Ltd., Netherlands) system with staggered oriented ridge (SOR) 3227 reactor chips (19.5 μL). The system is configured with five syringe pumps feeding a microreactor that is positioned onto a Peltier temperature control stage. FEP tubing (0.8 OD×0.25 mm ID, Dolomite Microfluidics) with 1 mL gastight glass syringes (Hamilton, Nev.) were used. The recipes for the reaction conditions are set onto the software ChemTrix GUI. For the scale up of the amidation and telescoped flow synthesis, PFA tubing ( 1/16″ OD×0.03″ ID, IDEX Health & Science) was coiled around Cu elbow joints (Home Depot) and placed in the oil bath at the desired temperature. All the microfluidic parts including unions, super-flangeless nuts, back-pressure regulators, and T-mixers were purchased from IDEX Health & Science.

5-Ethynyl-N-(4-((4-methylpiperazin-1-yl)methyl)-3-(trifluoromethyl)phenyl)nicotinamide (3). Amine 1 (151.63 mg, 0.55 mmol, 0.277 M), carboxylic acid 2 (80.92 mg, 0.55 mmol, 0.277 M), and DIPEA (287.4 μL, 1.65 mmol, 3 equiv.) were combined to make a final solution of 2 mL DMF that was subsequently loaded into a 1 mL Hamilton syringe mounted on Pump 1 of the Chemtrix reactor. A 2 ml solution of HATU (230.08 mg, 0.605 mmol, 1.1 equiv., 0.302 M) in DMF was prepared and loaded into another 1 mL Hamilton syringe and mounted on the Pump 2. The outlets of Pumps 1 & 2 were fed into a T-junction from where it enters the reactor holder containing the 3227 reactor chip (19.5 μL) placed onto the Peltier stage. Inlet 2 & 3 were blocked by blind plugs and the outlet of the reactor was channeled into the carousel unit. Waste material was collected for 3× residence times prior to collecting the sample for every condition for which recipes were made on the software. LC-MS was performed directly onto the crude reaction mixture by diluting it. For TLC analysis, reaction mixtures were diluted 1:1 with DCM. Product isolation for yield determination was achieved by collecting 500 μL of crude reaction mixture to which water was added directly until a slurry was observed as the product starts to precipitate. The product was then gathered by filtration and dried under vacuum overnight. TLC:R_{f} 0.5 (1:1 MeOH:Acetone). The ¹H-NMR and ¹³C-NMR, and HPLC data are provided in Supplementary Information.

5-((8-Amino-1,7-naphthyridin-5-yl)ethynyl)-N-(4-((4-methylpiperazin-1-yl)methyl)-3-(trifluoromethyl)phenyl)nicotinamide (5).

Continuous flow synthesis. A 4 mL DMF solution of Alkyne 3 (120.73 mg, 0.3 mmol, 0.075 M), Compound 4 (70.57 mg, 0.315 mmol, 0.0787 M), CuI (0.57 mg, 0.003 mmol, 0.75 mM) and pyrrolidine (75.12 μL, 0.915 mmol, 0.2287 M) was degassed by passing Ar for 30 minutes. It was loaded onto gastight glass syringe port 1 after filtering using a 0.2 μm PTFE syringe filter. Syringe 2 was loaded with 1 mol % of the catalyst (2.1 mg in case of PdCl₂(PPh₃)₂, 0.003 mmol) in DMF after purging it under Ar. [(t-Bu)₃PH]BF₄ was 3 mol % added when 3 mol % PdCl₂ or PdCl₂(MeCN)₂ was used as the catalyst in the syringe 2). A T-connector from the two syringe inlets after the check valve were fed into the Chemtrix reactor block mounted onto the heating Peltier stage heated to the desired temperature. Reactor chip 3227 (19.5 μL) was used for the runs. Blind plugs were placed on the second and third outlets. The final outlet was connected to the ultra-low volume back-pressure regulator followed by collection of the samples into the autosampler. The flow rate for S1 was 0.594 μL/min and for S2 was 0.186 μL/min thereby producing a residence time of 25 minutes. The flow rate for the pumps were changed to achieve residence times of 15, 25, 30, 40, and 50 minutes accordingly in the recipe. For analysis of the yield or conversion using LC-MS, 50 μL of the crude sample was collected and directly diluted in ACN to 50 μM and filtered using a 0.2 μm PTFE syringe filter. For the isolated yield determination, solutions of the crude product were extracted with 3× volume (i.e. volume of the crude solution collected) of DCM and washed with 1× water five times. The combined organic layers were washed with 1× volume sat. NH₄Cl, brine, dried over anhydrous Na₂SO₄, filtered and concentrated under reduced pressure. The crude mixture was purified by using column chromatography to obtain the desired product. (R_{f}: 0.2, 1:1 MeOH:Acetone). The ¹H-NMR and ¹³C-NMR matches with those reported in the literature.

Telescoped Continuous Flow Synthesis for HSN608 (5). A 30 mL stock solution in HPLC grade DMF containing 5-bromo-8-amino-1,7-naphthyridine (4) (436.9 mg, 1.95 mmol, 0.065 M), CuI (3.61 mg, 0.019 mmol, 0.632 mM), and pyrrolidine (457.57 μL, 5.57 mmol, 0.186 M) was prepared using a volumetric flask and the solution was then loaded into a 25 mL Hamilton syringe (labelled A) using a 0.2 μm syringe filter. PdCl₂(PPh₃)₂ (44 mg, 0.063 mmol, 2.09 mM) in 30 mL DMF, HATU (2489.4 mg, 6.54 mmol, 0.2182 M) in 30 ml DMF, a 30 mL solution containing carboxylic acid 2 (875.72 mg, 5.95 mmol, 0.1984 M), amine 1 (1626.68 mg, 5.952 mmol, 0.1984 M), and DIPEA (3110.36 μL, 17.85 mmol, 0.5952 M) in DMF were prepared and the respective solutions were loaded into Hamilton syringes labelled B, C, and D, respectively, before mounting onto the Harvard syringe pumps. The flow rates for syringes A, B, C, D were set as 14.26 μL/min, 4.326 μL/min, 4.45 μL/min and 4.45 μL/min. Other flow rates are listed in Table 3. Solutions of the crude product were extracted with 3× volume (i.e. volume of the crude solution collected) of DCM and washed with 1× water five times. The combined organic layers were washed with 1× volume sat. NH₄Cl, brine, dried over anhydrous Na₂SO₄, filtered and concentrated under reduced pressure. The crude mixture was purified by using column chromatography to obtain the desired product. (R_{f}: 0.2, 1:1 MeOH:Acetone).

Continuous flow synthesis of Amide Coupling for 3. 5-Ethynylnicotinic acid (1) (1.018 g, 6.925 mmol, 1 equiv.), 1.892 g of amine 2 (6.925 mmol, 1 equiv.), 3.618 mL (3 equiv.) DIPEA are added to a 25 mL volumetric flask that is then filled with DMF. HATU (2.870 g) was added to 25 mL volumetric flask filled with DMF. After a complete mixing of all the reagents in the flask, they are transferred to beaker before loading into Hamilton syringes. The coiled reactor was placed on a hot plate at 50° C. and equilibrated for 30 minutes. Prior to the reaction, 10 mL of DMF was flowed through the PFA tubing. The syringes with the respective solutions were mounted onto the Harvard syringe pump (Ultra) where a flow rate of 17.8 μL/min was set. The reaction solution was collected for 3 times residence time (i.e., 60 minutes for equilibration), before switching to collect the crude reaction solution in a 50 mL Falcon tube. Water was added to the crude reaction solution until precipitation was observed. The precipitate was filtered using a fritted funnel to obtain 2.260 g of the desired product (Yield=83%).

Supporting Information. Detailed experimental procedures for high throughput experimentation, ESI-MS, DESI-MS, batch synthesis, continuous flow synthesis, telescoped synthesis, DoE analysis, ¹H-NMR & ¹³C-NMR, and spectrometric data are available in Supporting Information. This is available free of charge via the Internet http://pubs.acs.org.

### Abbreviations

HTE, high throughput experiment; DoE, design of experiments; DESI-MS, desorption electrospray ionization-mass spectrometry; FLT-3, fms-like tyrosine kinase 3; OVAT, one variable at a time; CHRIS, chemical reaction integrated screening software; AML, acute myeloid leukemia; TLC, thin layer chromatography; LC-MS, liquid chromatography mass spectrometry; ESI-MS, electrospray-ionization mass spectrometry; NMR, nuclear magnetic resonance; CFI, coiled flow inverter; R_{T}, residence time; HATU, O-(7-Azabenzotriazol-1-yl)-N,N,N′,N′-tetramethyluronium hexafluorophosphate; TMPH, 2,2,6,6 tetramethylpiperidine; TBAPF₆, tetrabutylammoniumhexafluorophosphate; Pd, Palladium; Cu, Copper; DIPEA, Diisopropyl ethylamine; TEA, Triethylamine; TMEDA, N,N,N′,N′-Tetramethylethylenediamine; K₃PO₄, Potassium phosphate; ANOVA, analysis of variance; K₂CO₃, Potassium carbonate; PdCl₂, Palladium (II) chloride; [(t-Bu)₃PH]BF₄, Tri-tert-butylphosphonium tetrafluoroborate; PdCl₂(PPh₃)₂, Bis(triphenylphosphine)palladium(II) dichloride; Pd(amphos)Cl₂, [Bis(di-tert-butyl(4-dimethylaminophenyl)phosphine)dichloropalladium(II)]; XPhos Pd G3, (2-Dicyclohexylphosphino-2′,4′,6′-triisopropyl-1,1′-biphenyl)[2-(2′-amino-1,1′-biphenyl)]palladium(II) methanesulfonate; PdCl₂(MeCN)₂, Bis(acetonitrile)palladium dichloride; Pd(OAc)₂, Palladium(II) Acetate; DMF, N,N′Dimethylformamide; EtOH, Ethanol; ACN, Acetonitrile; DMAc, N,N′Dimethyacetamide; NMP, N-methylpyrrolidone; PFA, Perfluoroalkoxy, SOR, Staggered Oriented Ridge.

Those skilled in the art will recognize that numerous modifications can be made to the specific implementations described above. The implementations should not be limited to the particular limitations described. Other implementations may be possible.

1. A continuous flow Sonogashira coupling synthesis method to prepare a compound of Formula I, wherein the method comprises:
providing a first flow comprising an aryl or a heteroaryl halide compound of Formula II, a base, and an optional Cu(I) halide;
providing a second flow comprising a Pd(II)-based catalyst;
providing a third flow comprising an aryl or heteroaryl alkyne of Formula III;
wherein the first flow, second flow and the third flow are mixed and fed into a flow reactor to carry out a Sonogashira coupling reaction to provide the compound of Formula I,
wherein the Formula I is 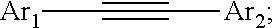
wherein the Formula II is Ar₁—X, where X is Cl, Br, or I;
wherein the Formula III is 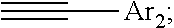
wherein Ar₁ and Ar₂ are each independently an optionally substituted monocyclic or bicyclic aryl or heteroaryl.

2. The method of claim 1, wherein the Pd(II)-based catalyst comprises PdCl₂(PPh₃)₂, PdCl₂(MeCN)₂, XPhos Pd G3, PdCl₂, allyl palladium(II) chloride dimer, Pd(amphos)Cl₂, or Na₂PdCl₄.

3. The method of claim 1, wherein the base comprises pyrrolidine, K₂CO₃, Cs₂CO₃, 2,2,6,6 tetramethyl piperidine, tetrabutylammonium hexafluorophosphate, 1,4-diazabicyclo[2.2.2]octane (DABCO), quinuclidine, triethyl amine (TEA), N,N-diisopropylethylamine (DIPEA), tetrabutylammonium acetate, or piperidine.

4. The method of claim 1, wherein the optional Cu(I) halide is CuI.

5. The method of claim 1, wherein each flow comprises a solvent, wherein the solvent comprises N-methyl-2-pyrrolidone (NMP), dimethylformamide (DMF), DMF/H₂O, ethanol, dimethylacetamide (DMAC), N, N′-dimethylpropyleneurea (DMPU), 1,4-dioxane, tetrahydrofuran (THF), or 2-methyl tetrahydrofuran.

6. The method of claim 1, wherein each flow comprises an optional ligand, wherein the ligand comprises [(t-Bu)₃PH]BF₄, t-Bu₃P, RuPhos, Brett Phos, or XPhos.

7. The method of claim 1, wherein one or more hydrogen of Ar₁ and/or Ar₂ can be independently substituted by —NH₂, —CN, —CF₃, —F, Cl, —Br, —I, —OH, or an optionally substituted amide, sulfonamide, or urea.

8. The method of claim 1, wherein the flow reactor is a plug flow reactor, segmented flow reactor, a microreactor, coiled tubing reactor, coiled flow inverter (CFI) reactor, or continuous stirred tank reactor (CSTR) in a flow configuration.

9. The method of claim 1, wherein the flows are mixed using T-mixers, Y-mixers, static-mixers, ultrasonic mixers, staggered oriented ridge mixers, zig-zag mixers, packed bed mixers, CFI mixers, or paddle mixers in a CSTR in a flow configuration.

10. A continuous flow Sonogashira coupling synthesis to prepare a compound of Formula A, wherein the method comprises:
providing a first flow comprising an aryl or a heteroaryl halide compound of Formula B, a base, and an optional Cu(I) halide;
providing a second flow comprising a Pd(II)-based catalyst;
providing a third flow comprising an aryl or heteroaryl carboxylic acid of Formula C, an aryl or heteroaryl amine of Formula D; and
providing a fourth flow comprising an amide coupling reagent,
wherein said third flow and fourth flow are mixed and fed into a first reactor to carry out an amide coupling reaction to provide an amide compound of Formula E, and the formed amide compound of Formula E is released from the first reactor to form a fifth flow,
wherein the first flow, second flow and the fifth flow are mixed and fed into a second reactor to carry out a Sonogashira coupling reaction to provide the compound of Formula A,
wherein the Formula A is: 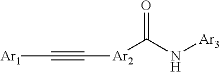
wherein the Formula B is Ar₁—X, X is Cl, Br, or I;
wherein the Formula C is: 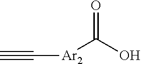
wherein the Formula D is Ar₃—NH₂,
wherein the Formula E is: 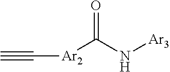
wherein Ar₁, Ar₂, Ar₃ are each independently an optionally substituted monocyclic or bicyclic aryl or heteroaryl.

11. The method of claim 10, wherein the Pd(II)-based catalyst comprises PdCl₂(PPh₃)₂, PdCl₂(MeCN)₂, XPhos Pd G3, PdCl₂, allyl palladium(II) chloride dimer, Pd(amphos)Cl₂, or Na₂PdCl₄.

12. The method of claim 10, wherein the base comprises pyrrolidine, K₂CO₃, Cs₂CO₃, 2,2,6,6 tetramethyl piperidine, tetrabutylammonium hexafluorophosphate, 1,4-diazabicyclo[2.2.2]octane (DABCO), quinuclidine, triethyl amine (TEA), N,N-diisopropylethylamine (DIPEA), tetrabutylammonium acetate, or piperidine.

13. The method of claim 10, wherein the optional Cu(I) halide is CuI.

14. The method of claim 10, wherein each flow comprises a solvent, wherein the solvent comprises N-methyl-2-pyrrolidone (NMP), dimethylformamide (DMF), DMF/H₂O, ethanol, dimethylacetamide (DMAC), N, N′-dimethylpropyleneurea (DMPU), 1,4-dioxane, tetrahydrofuran (THF), or 2-methyl tetrahydrofuran.

15. The method of claim 10, wherein each flow comprises an optional ligand, wherein the ligand comprises [(t-Bu)₃PH]BF₄, t-Bu₃P, RuPhos, Brett Phos, or XPhos.

7. (canceled)

16. The method of claim 10, wherein the flow reactor is a plug flow reactor, segmented flow reactor, microreactor, coiled tubing reactor, or coiled flow inverter (CFI) reactor.

17. The method of claim 10, wherein the flows are mixed using T-mixers, Y-mixers, static-mixers, ultrasonic mixers, staggered oriented ridge mixers, zig-zag mixers, packed bed mixers, or CFI mixers.

18. The method of claim 10, wherein Ar₁ is 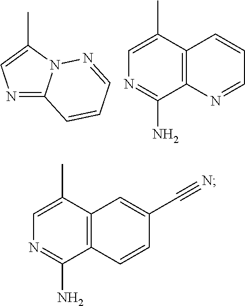
Ar₂ is a pyridinyl ring, and
Ar₃ is

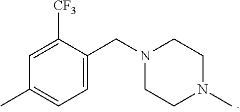

19. The method of claim 10, wherein the compound of Formula A is selected from:

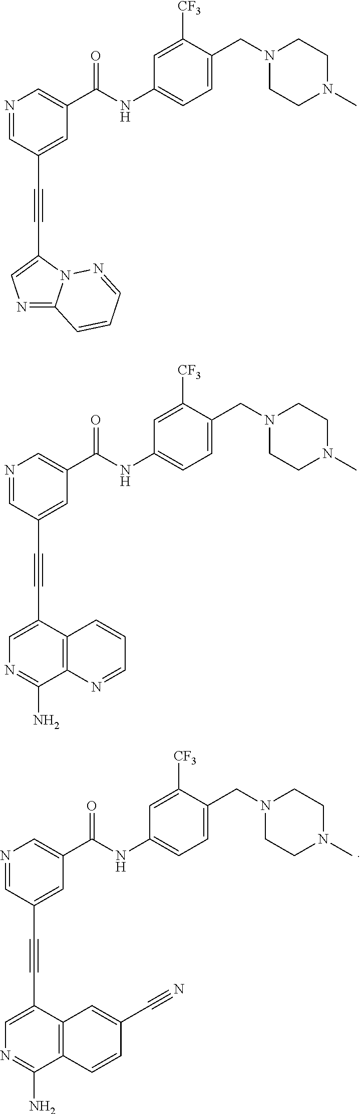 [SMILES: CN1CCN(Cc2ccc(NC(=O)c3cncc(C#Cc4cnc5cccnn45)c3)cc2C(F)(F)F)CC1 ; CN1CCN(Cc2ccc(NC(=O)c3cncc(C#Cc4cnc(N)c5ncccc45)c3)cc2C(F)(F)F)CC1 ; CN1CCN(Cc2ccc(NC(=O)c3cncc(C#Cc4cnc(N)c5ccc(C#N)cc45)c3)cc2C(F)(F)F)CC1]

20. The method of claim 10, wherein the compound of Formula A is:

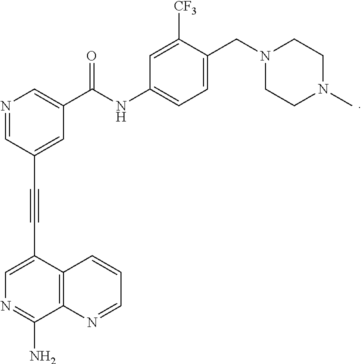 [SMILES: CN1CCN(Cc2ccc(NC(=O)c3cncc(C#Cc4cnc(N)c5ncccc45)c3)cc2C(F)(F)F)CC1]

21. The method of claim 10, wherein one or more hydrogen of Ar₁, Ar₂ and/or Ar₃ can be independently substituted by —NH₂, —CN, —CF₃, —F, Cl, —Br, —I, —OH, or an optionally substituted amide, sulfonamide, or urea.
